(19) United States
(12) Patent Application Publication
(10) Pub. No.: US 2022/0175134 A1
(43) Pub. Date: Jun. 9, 2022

(54) SENSORY MAT DESKTOP COVER AND METHOD

(71) Applicant: Ann M. Adkins, Daniels, WV (US)

(72) Inventor: Ann M. Adkins, Daniels, WV (US)

(21) Appl. No.: 17/545,912

(22) Filed: Dec. 8, 2021

Related U.S. Application Data
(60) Provisional application No. 63/122,883, filed on Dec. 8, 2020.

(51) Int. Cl. A47B 41/00; A61M 21/00; A47B 13/08

(52) U.S. Cl. CPC A47B 41/00; A61M 21/00; A47B 2230/0011; A61M 2021/0022; A47B 2200/0084; A47B 13/08

(57) ABSTRACT

A school desktop cover for special needs students that is easy to install and remove. The cover can be strapped about (and beneath) a typical elementary school desktop so as to not interfere with the regular use of the desktop and/or the positioning of a student's legs there beneath. Full desktop varieties are intended as well as partial desktops and mat strip/straps. Each version contains a plurality of sensory workstation activities. A portable version for transporting between classrooms, or for the student to use while eating or at home is also anticipated. A method of use is also claimed.

### REFERENCE TO RELATED APPLICATIONS

This application is a perfection of U.S. Provisional Application No. 63/122,883, filed on Dec. 8, 2020, the disclosure of which is fully incorporated by reference herein.

### STATEMENT OF GOVERNMENT INTEREST

Not Applicable

### STATEMENT AS TO RIGHTS TO INVENTIONS MADE UNDER FEDERALLY SPONSORED RESEARCH OR DEVELOPMENT

Not Applicable.

### NAMES OF THE PARTIES TO A JOINT RESEARCH AGREEMENT

Not Applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable.

### STATEMENT REGARDING PRIOR DISCLOSURES BY THE INVENTOR OR A JOINT INVENTOR

Not Applicable.

### BACKGROUND OF THE INVENTION

### (1) Field of the Invention

The purpose behind these various desktop cover designs is to accommodate special needs students who are included in the regular education classroom. Such students often struggle with the demands of the class and length of the class period. This invention will provide such students with physical and mental exercises to work on during classroom downtimes and the like. By downtimes, it is meant the Student's Downtime, rather than that of the teacher, or teacher's aide. These are meant to address, for certain special students, the times that such students need to be occupied/preoccupied/focused on sensory stimulations—especially when the teacher may be lecturing. These are especially perceived as desk mats for such a student to fidget with and help them stay seated, focused and busy.

### BRIEF SUMMARY OF THE INVENTION

This invention addresses a school desktop cover that is easy to install and remove as needed. The cover can be strapped about (and beneath) a typical elementary school desktop so as to not interfere with the regular use of the desktop and/or the positioning of a student's legs there beneath. Full desktop varieties are intended as well as partial desktops and activity mat strips (or deskstraps). A more portable version (for transporting between classrooms, between stations in the same classroom, or for the student to use while eating or while learning from home) is also anticipated.

Each cover would be made from durable materials, including canvas and the like, and include a plurality of activities thereon. Many of the illustrated representative covers include at least 7 separate and distinct sensory exercise activities (or “stations”). In future variations, each desktop cover may include an ability to remove/replace, i.e., change in and out, various activity stations on the same cover.

Another aspect of this invention entails an improved method for teaching special needs students by providing them with sensory mat desktop covers like those shown and described herein.

### BRIEF DESCRIPTION OF THE DRAWING(S) AND PHOTOGRAPH(S)

In the accompanying drawings, some mat dimensions are provided but they are merely suggestive or representative for sample mat versions. Further features, objectives and advantages will become clearer when reviewing the following description of preferred embodiments made with reference to the accompanying drawings and photographs in which:

FIG. 1 is a top perspective photograph of one embodiment of sensory mat cover before it would be installed about a student's desk according to this invention;

FIG. 2 is a top plan view of a desktop cover, with preferred dimensions, before workstations are added thereto;

FIG. 3 is a rear plan view showing a first embodiment of cover installed on a student's desk;

FIG. 4 is a right side view of the same desktop cover from 3;

FIG. 5 is a top plan view showing the same cover from FIGS. 3 and 4 with specifically itemized activity areas/substations;

FIG. 6 is a bottom plan view showing the underside of a desk to which the invention has been installed;

FIG. 7 is a top perspective photograph showing a second embodiment of sensory mat for only partially covering a student's desk;

FIG. 8 is a top plan view of the partial desktop cover from FIG. 7 with one set of representative preferred dimensions;

FIG. 9 is a rear plan view showing the partial desktop cover from FIGS. 7 and 8 installed on about half of a student's desktop;

FIG. 10 is a right side view of the same partial desktop cover from 9;

FIG. 11 is a top plan view showing the same partial desktop cover from FIGS. 7 through 10 with specifically itemized activity areas/substations;

FIG. 12 is a bottom plan view showing the underside of a desk onto which the partial desktop cover version has been installed;

FIG. 13 is a top perspective photograph showing a third embodiment of this invention comprising a thin activity sensory strip (or deskstrap) for adding to a student's desk;

FIG. 14 is a top plan view of the activity strip desktop cover from FIG. 13 with one set of representative preferred dimensions;

FIG. 15 is a top plan view of a whole activity strip desktop cover with representative activities thereon and attachment strips extending outwardly from both lateral sides with specifically itemized activity areas/substations;

FIG. 16 is a top plan view of a fourth version of “portable” activity sensory map workstation for use on any desktop or lunch table with one set of representative preferred dimensions therefor;

FIG. 17 is a top plan view of the portable activity sensory map workstation from FIG. 16 with specifically itemized activity areas/substations; and

FIG. 18 is a bottom plan view of the same workstation from FIG. 17 with one representative set of slide prevention means added thereto.

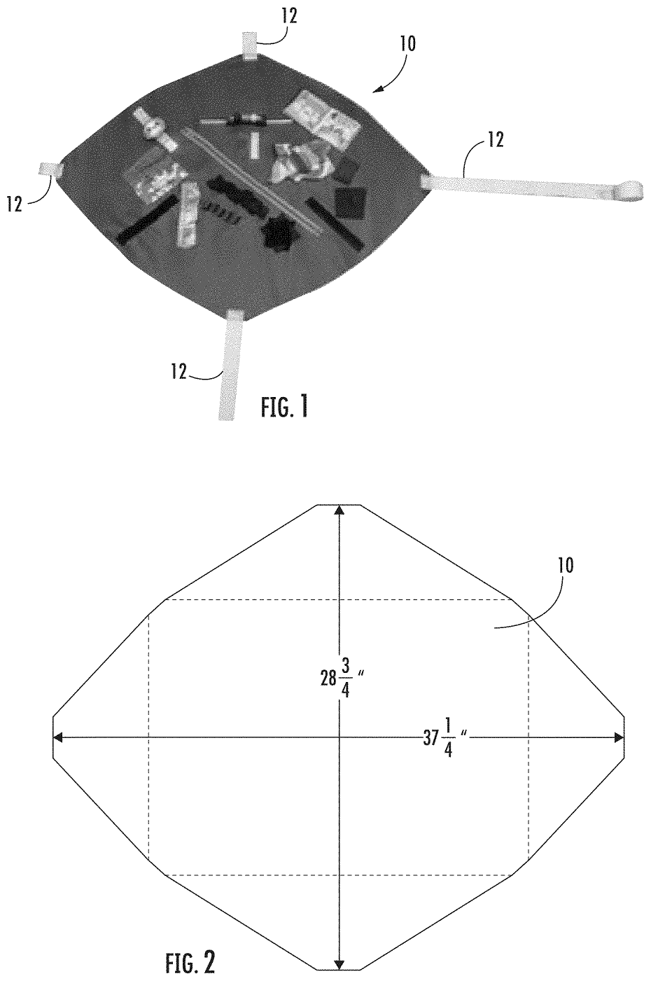

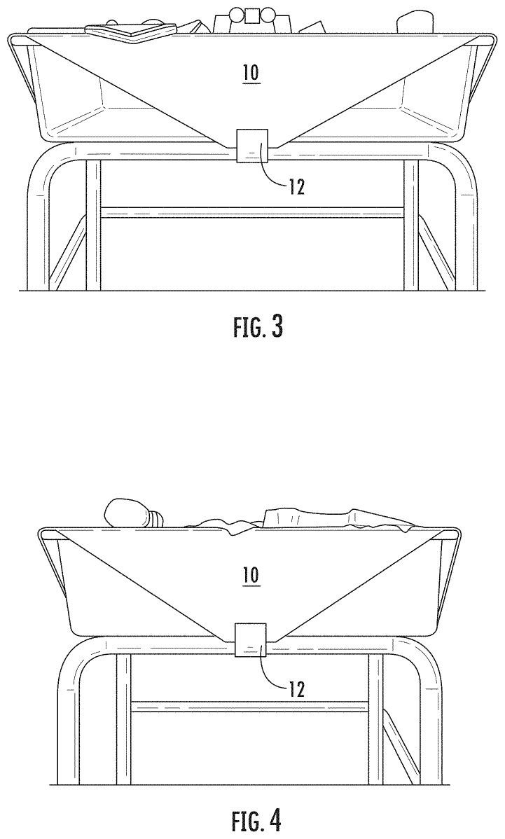

### DETAILED DESCRIPTION OF THE INVENTION

The following detailed description of implementations consistent with the present invention refers to the accompanying photographs and drawings. The same reference numbers in different drawings may identify the same or similar elements—commonly numbered though in the next hundred series. Also, the following detailed description does not limit the invention. Instead, the scope of the invention is defined by the appended claims and equivalents.

Teacher downtimes are defined to include: times between class instruction/or lesson plan times, snack or lunch times and during classroom OR classroom station transitions.

Construction of the design—The construction of these various desktop cover configurations was designed with the intended end users in mind, both teacher AND student. The main body of the cover component should be made from a durable upholstery fabric that is easy to clean. All of the sensory workstations (or “manipulates”) added to the main body cover component should either be glued on, sewn with heavy upholstery thread or both glued and sewn. Cover corners should be especially secured (hence the logic behind locating securing straps at these corners), i.e., for resisting being picked at/picked off by the student sitting there.

The covers will be easy to disinfect. The straps secure it in place to prohibit removal by the student.

Alternatives to the current design could include, but is not limited to the following:

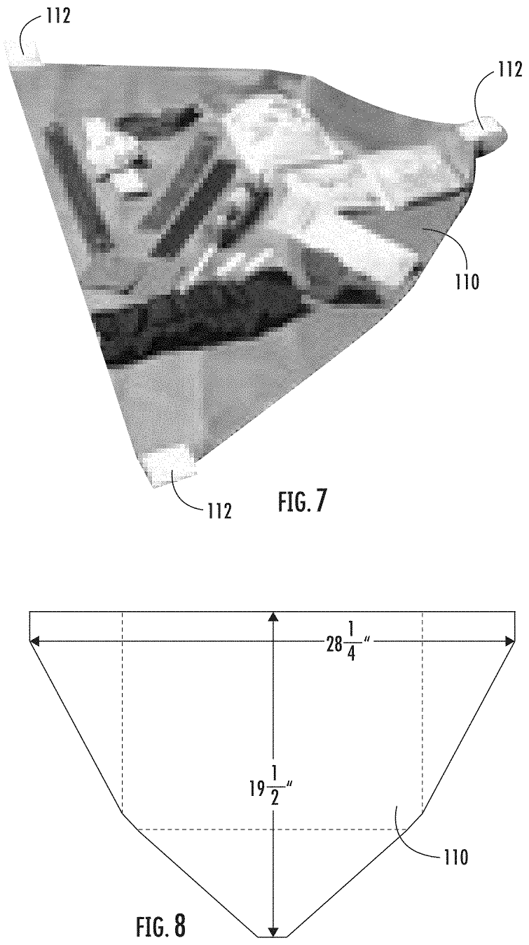

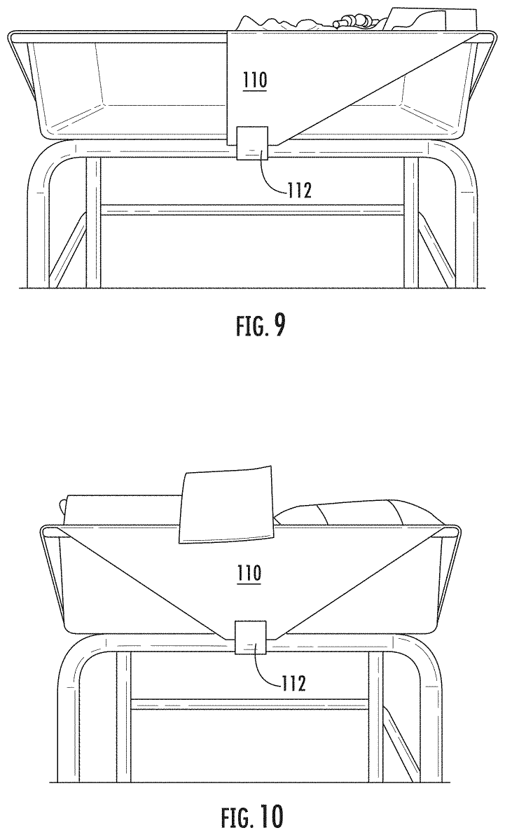

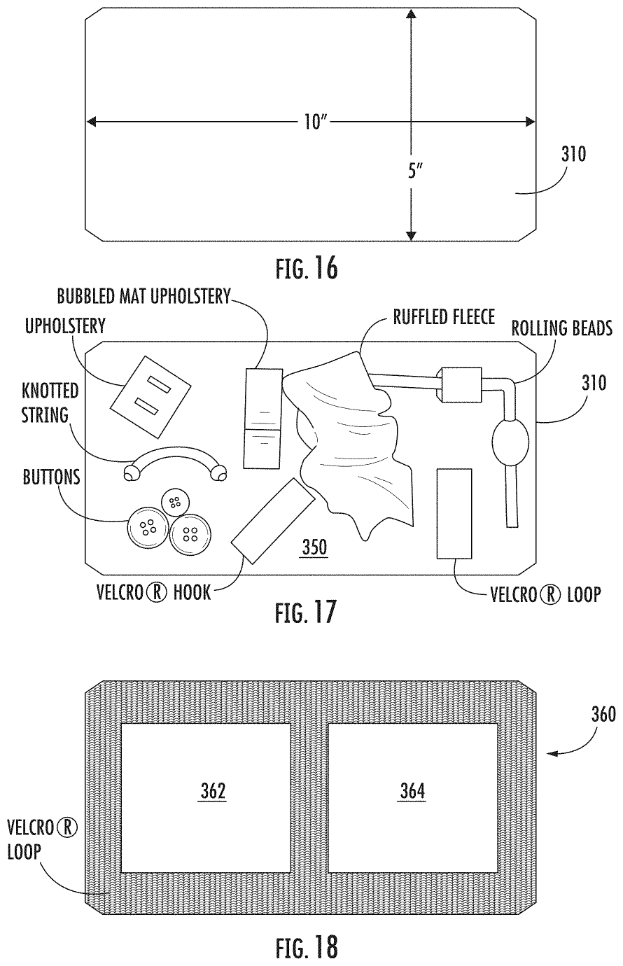

1. A section of the cover could be left vacant for work or assignments to be completed by the student on a flat surface as per FIGS. 7 through 12. Alternately, there can be provided a “temporary” flat overlay area for the student that can easily install on and be removed from (by an adult) these mat desktops. See especially, accompanying FIGS. 16 to 18.
2. The size of the cover could vary depending on the desk size and/or shape.
3. Different sensory manipulatives could be attached and/or designed.

The inventor further envisions the following:

1. Improved straps made of nylon with double ring closures or single adjust squeeze side buckles as per accompanying FIGS. 13 to 15. The goal is to NOT interfere with the student's legs positioned beneath such mat desktop covers yet still provide them will safe and secure attachment to a desktop while needed for daily classroom activities.
2. Adapting the cover for use in a special education classroom.
3. Using a sturdier or improved fabric for the base mat.
4. Incorporating different (student customized) sensory items for varied sensory needs.
5. Including a plurality of activity/stations on a pseudo lazy Susan turntable (not shown) to allow the adult in charge to rotate which activities will be placed directly in front of the student for a given activity time period.

### EXAMPLES

Four representative varieties of sensory mat covers are depicted in the accompanying photographs and line drawings.

### Set 1

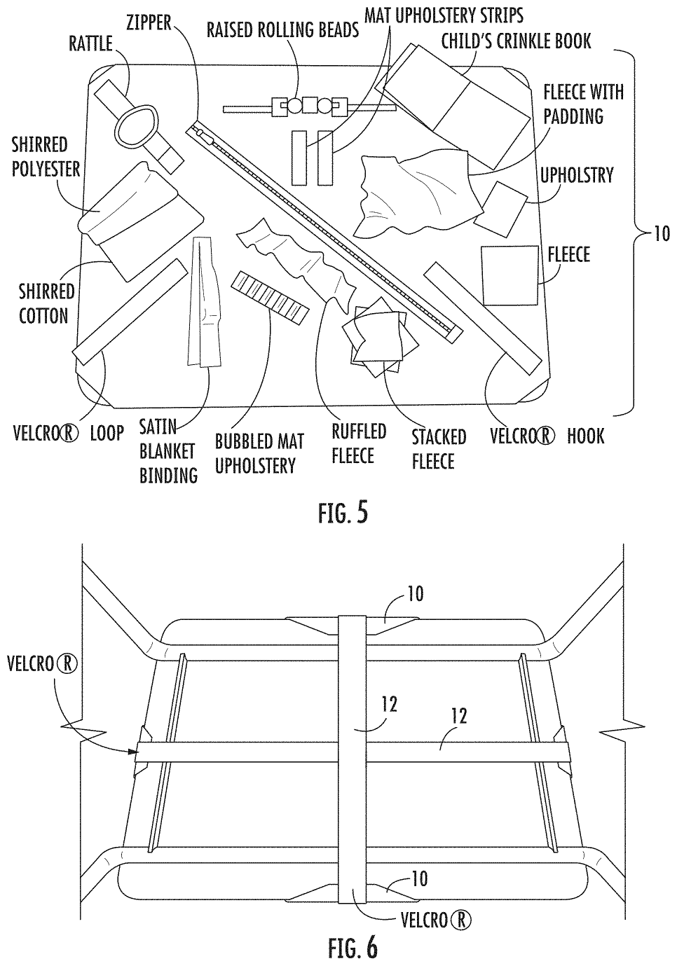

The diamond-shaped mat, generally 10, with attachment straps 12, shown at FIGS. 1 and 5 includes the following sensory activities as best labelled in accompanying

FIG. 5, may include but not be limited to: a. a pair of zipper strips, b. a soft-sided animal book, c. sections of various tactile materials, d. an anchored wavy cloth, e. a section of hook-and-loop (or Velcro ® brand) tape; f. a series of knotted cloth samples; g. a series of intermittently spaced loops; and h. a set of rotatable beads on a permanently raised rod.

Though not shown in FIGS. 1 and 5, each mat may further include a pillowy region on which the student may temporarily rest his/her arm and/or head. Alternately, each mat may include a specially assigned section to serve as a work area on which the student may perform other (i.e., more scholastically-based) classroom activities.

### Set 2

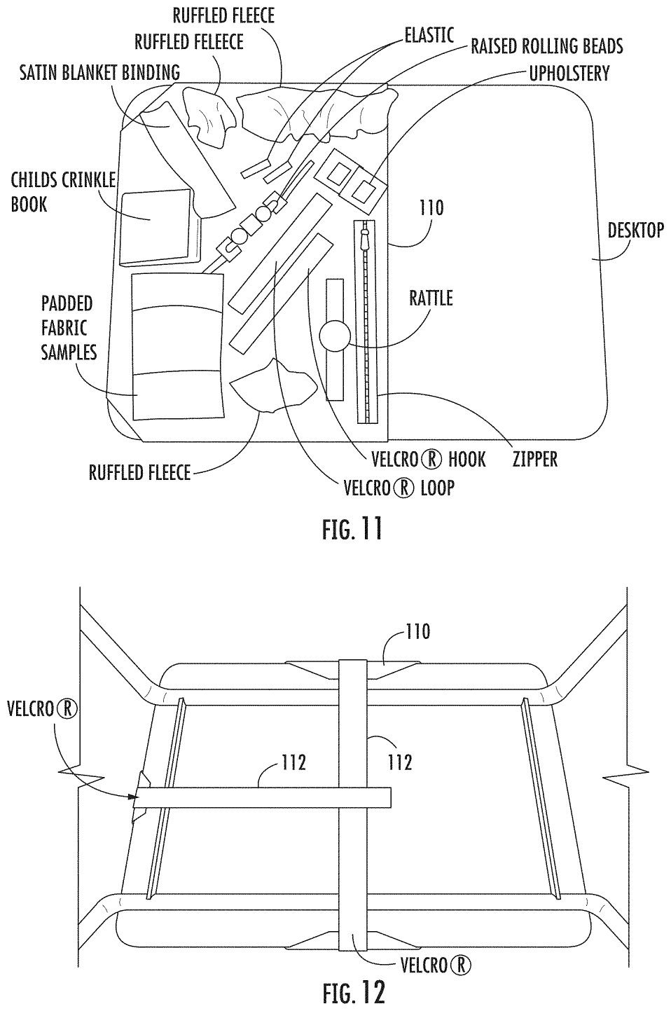

The mat 110 shown at FIGS. 7 and 11, with attachment straps 112, includes several of the same activities/manipulates as per FIGS. 1 and 5, on a shorter, different overall deskmat layout , i.e., one that takes up just about half (or possibly even less) of the child's main desktop work area.

For specifics on the various “representative” substations or activity “workstations” of this second embodiment, see the itemized listing at FIG. 11. They may particularly include: series of round and cubed blocks on a string; and a fluffy, floaty cloth for the student to rub about with his/her fingers. If attachment means can be addressed, perhaps even fidget spinner components may be added to one or more of these desk mat versions. Set 3

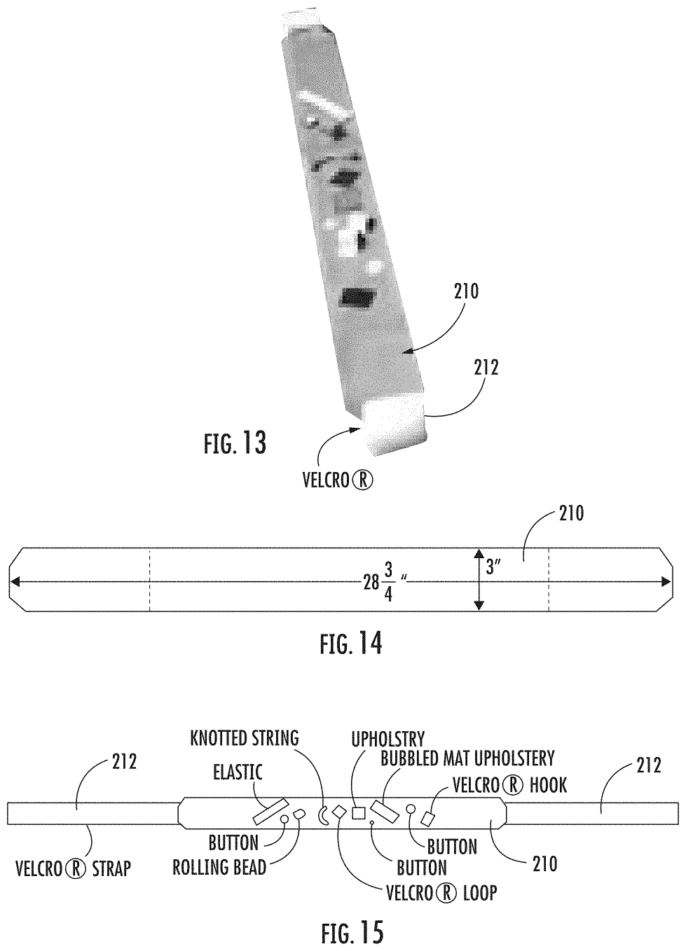

This third version of mini mat strip, or deskstrap 210, with at least partially wrapping around attachment end straps 212, is meant to be much less obtrusive but also more “portable”—for when the same student(s) travel about . . . in and out of multiple classrooms. In addition to some of the same activities as the first two variations, once more commonly numbered though in the next hundred series, this third variety of shortened activity mat deskstrap 210 (see, especially the labelled list of stations at FIG. 15) includes: a plurality of various sized square shapes. These shapes would be purposefully, permanently attached at their middles to the main mat strap body 210 for the student to perform finger exercising about the unstuck square perimeters.

### Set 4

For the fourth variation of this invention, consider a representative, portable mat 310, like that shown in FIGS. 16-18, and as more fully itemized at FIG. 17. The top to main mat body 310 has an activity area 350 with multiple stations including but not limited to: ruffled fleece, bubbled mat upholstery, rolling beads, Velcro® loop strips, Velcro® hook strips, buttons, knotted strings and other upholstery fabric section as duly labelled. It is to be understood that some of the various activity areas of this mat version may be used to substitute for, OR further supplement the activity areas of these earlier workstation versions, especially when attachments to a given main body mat are meant to be temporary, for easier swap outs by a supervising adult.

For better securing the “portable” mat version to a student's desk, the underside 360 of portable mat body 310 may include large sections of slide resistant, rubberized squares 362, 364, or alternative temporary security materials employing one or more sections of Velcro® hook and loop combinations 370, especially about the edges (and perhaps middle sections) of these mats.

Still other individualized activity stations (not pictured) may include: Pop-its, spinners and toggle areas.

Having described the best modes currently known for practicing this system and method, it is to be understood that the scope of this invention may be further described by the attached claims.

### SEQUENCE LISTING

Not applicable

What is claimed is:

1. A sensory mat desktop cover for a special education student comprising: a main base mat; means for securing the main base mat about at least a portion of the student's desktop; and a plurality of sensory exercise activity areas/stations adapted for at least temporarily attaching to the main base mat.

2. The sensory mat desktop cover of claim 1, which further includes an assigned student resting area.

3. The sensory mat desktop cover of claim 1, which further includes an assigned student work area.

4. The sensory mat desktop cover of claim 3 wherein the student work area is in a central region of the main base mat closest to where the student sits.

5. The sensory mat desktop cover of claim 1 wherein the main base mat is made from an easily cleanable material selected from the group consisting of canvas, Naugahyde and vinyl.

6. The sensory mat desktop cover of claim 1 wherein two or more of the activity areas/stations are only temporarily secured to the main base mat for subsequent removal and replacement with other activity areas/stations.

7. The sensory mat desktop cover of claim 1 wherein securing means include a pair of criss-crossing canvas straps.

8. The sensory mat desktop cover of claim 7 wherein said crisscrossing straps attach to one another, beneath the desktop, using at least one of: a buckle system, a stringed tie system, a series of clips and a system of hook-and-loop (or Velcro®) strips.

9. The sensory mat desktop cover of claim 1, which covers a majority of the student's desktop.

10. The sensory mat desktop cover of claim 1, which covers about half or less of the student's desktop.

11. The sensory mat desktop cover of claim 1 wherein the main base mat consists of one or more wrap around desk straps for securing above and below the student's desktop.

12. A portable, sensory mat for a special education student comprising: a main base mat body; means for temporarily securing the main base mat body to a flat surface in front of the student; and a plurality of sensory exercise activity areas/stations adapted for at least temporarily attaching to the main base mat body.

13. The portable, sensory mat of claim 12 wherein the plurality of sensory exercise activity areas/stations are permanently attached to the main base mat body.

14. The portable, sensory mat of claims 12, which may be positioned in front of the student while eating.

15. The portable, sensory mat of claims 12, which may be positioned in front of the student while at school or at home.

16. A method for assisting a teacher/teacher aide of a student with special sensory needs comprises:
(a) providing the student desks with a sensory mat desktop for occupying the student's sensory needs during teacher/teacher aide downtimes, said mat desktop having a plurality of activity areas/stations;
(b) at least temporarily securely installing the sensory mat desktop to at least a portion of the student's desktop.

17. The method of claim 16, which further includes:
(c) periodically changing out activity areas/stations on the student's sensory mat desktop cover.

18. The method of claim 16 wherein the sensory mat desktop cover covers most of the student's desktop.

19. The method of claim 16 wherein the sensory mat desktop cover covers about half or less of the student's desktop.

20. The method of claim 16 wherein the sensory mat desktop cover consists of one or more wrap around desk straps for securing above and below the student's desktop.
